(19) United States
(12) Patent Application Publication
(10) Pub. No.: US 2026/0124670 A1
(43) Pub. Date: May 7, 2026

(54) DEVICE FOR BENDING AND SECURING SURGICAL NEEDLES AND RELATED METHODS AND SYSTEMS

(71) Applicant: University of Iowa Research Foundation, Iowa City, IA (US)

(72) Inventor: Robert Bowers, Iowa City, IA (US)

(73) Assignee: University of Iowa Research Foundation, Iowa City, IA (US)

(21) Appl. No.: 19/379,266

(22) Filed: Nov. 4, 2025

Related U.S. Application Data
(60) Provisional application No. 63/716,022, filed on Nov. 4, 2024.

(51) Int. Cl. B21G 1/00; A61B 17/34

(52) U.S. Cl. CPC B21G 1/003; A61B 17/3417; A61B 2017/347

(57) ABSTRACT

Disclosed herein are implementations of a device, and associated methods and systems, for bending and securing a surgical needle to a predetermined angle relative to the base of the needle. One feature of various disclosed implements of the invention is a two-part construction that provides additional needle stability after bending.

### CROSS-REFERENCES & RELATED APPLICATIONS

This application claims priority to U.S. Provisional Application No. 63/716,022 filed Nov. 4, 2024, and entitled “DEVICE FOR BENDING AND SECURING SURGICAL NEEDLES AND RELATED METHODS AND SYSTEMS,” which is hereby incorporated by reference in its entirety under 35 U.S.C. § 119(e)

### TECHNICAL FIELD

The disclosure relates to surgical tools generally and bending surgical needles specifically.

### BACKGROUND

Various surgical tasks require a surgical needle, such as may be attached to a syringe, to be bent to some angle relative to the base of the needle. While surgical needles of various angles have applications in various surgical settings, they are of particular value in dental procedures, as the tight confines of the mouths of patients may make it difficult to align a straight needle with the injection sight properly. An important consideration in bending surgical needles is to ensure the needle is not bent at the hub of the needle, as bending at or near the hub may increase the risk of separation or breakage. Providing sufficient stability during and after the process of bending a needle is important to ensure safety and accuracy of subsequent injections.

There is a need in the art for a device, and accompanying methods and systems, that may allow for reliable, stable, and consistent bending of a surgical needle.

### BRIEF SUMMARY OF THE INVENTION

The various implementations included in this disclosure are directed to a device to bending and securing a surgical needle to some angle relative to the base of the needle, and accompanying methods and systems. Certain implementations may include devices for ensuring the consistent bending of a needle, while other implementations may also include devices to secure the needle at a particular angle once bent.

While multiple implementations are disclosed, still other embodiments of the disclosure will become apparent to those skilled in the art from the following detailed description, which shows and describes illustrative implementations of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1 is a diagram of the base of the device, according to one implementation.

FIG. 2A is a diagram of the base of the device viewed from below, according to one implementation.

FIG. 2B is a diagram of the base of the device viewed from above, according to one implementation.

FIG. 3 is a diagram of the brace of the device view from the front, according to one implementation.

FIG. 4 is a diagram of the brace of the device view from the rear, according to one implementation.

FIG. 5 is a diagram of the assembled device, according to one implementation.

FIG. 6 is a diagram of the handle of the device viewed from the front, according to one implementation.

FIG. 7 is a diagram of the handle of the device viewed from the rear, according to one implementation.

FIG. 8 is a guide for a method of use of the device, according to one implementation.

### DETAILED DESCRIPTION

Disclosed herein are implementations of a device, and associated methods and systems, for bending and securing a surgical needle to a predetermined angle relative to the base of the needle.

As used herein, the term “complement”, when referring to the relationship between different components or surfaces, means the components or surfaces are shaped to fit together securely, such as one component securely fitting inside another.

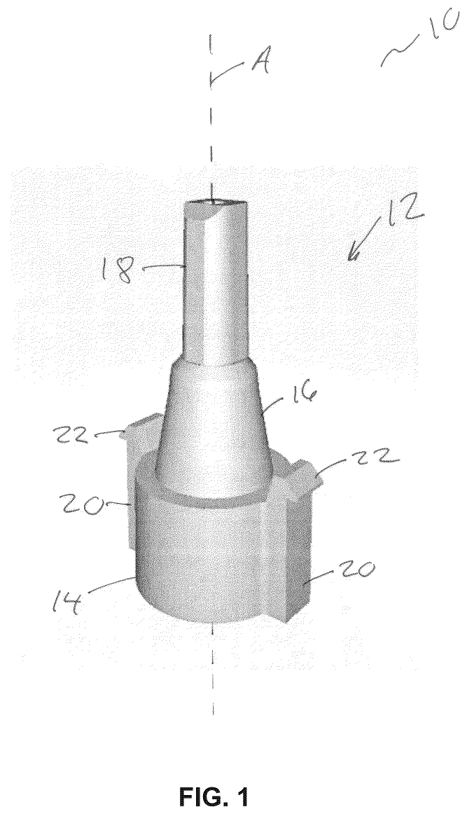

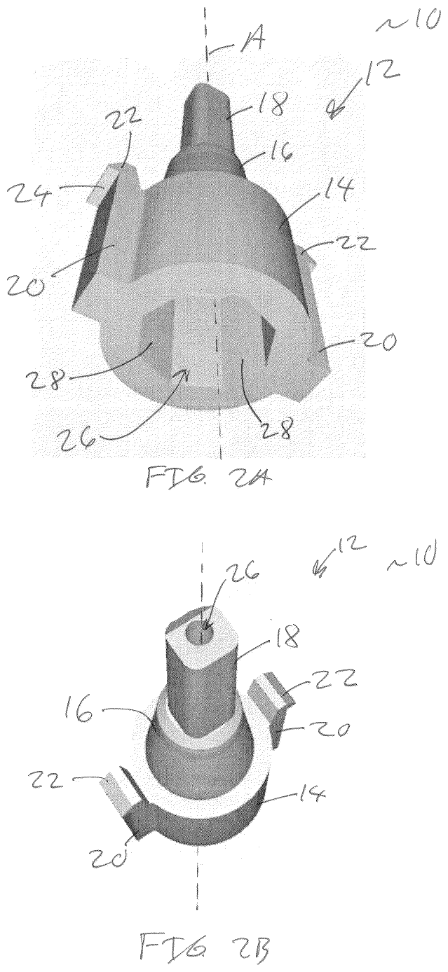

FIG. 1 shows a base 12 that is a component of the bending device 10 in various implementations. The specific geometry of the base 12 described herein is a single implementation of the base 12. As would be understood, variations to the specifics of the geometry of each component, such as the base 12 and others, may be done without deviating from the scope of the bending device 10 as a whole.

The base 12 may be made of a proximal body 14 with a medial body 16 extending from the proximal body 14 and aligned axially with the proximal body 14. In certain implementations, the proximal body 14 may be generally cylindrical in shape, although other shapes are, of course, possible. Likewise, the medial body 16, in certain implementations, may be generally conical in shape, although other shapes are possible. The base 12 may also have a distal body 18, which may have chamfered sides, extending from the medial body 16 on the opposite side of the medial body 16. The distal body 18 may also be aligned axially with the proximal body 14 and medial body 16. In certain implementations, the distal body 18 may be generally cuboid in shape in the general shape of a rectangular prism, although other shapes are, of course, possible. The aligned axes of the proximal body 14, medial body 16, and distal body 18 may align with a central axis A.

The proximal body 14, in various implementations, may have one or more wings 20 that may be protrusions extending outwardly from the cylindrical main body. Each wing 20 may have a tab 22 constructed with at least one substantially flat surface 24 (shown in FIG. 2A) substantially orthogonal to the central axis A.

Turning to FIGS. 2A and 2B, the base 12 may have a lumen 26 disposed through it, generally aligned with and centered on the central axis A. In various implementations, the lumen 26 may change in diameter along the length of the base 12. In certain implementations, the lumen 26 may be relatively small in the distal body 18 and relatively large in the proximal body 14. The lumen 26 in the medial body 16 may expand to transition between the small diameter in the distal body 18 and the proximal body 14.

In various implementations, the lumen 26 may have a plurality of planar surfaces 28 around its circumference, optionally in the portion of the lumen 26 disposed in the proximal body 14. The plurality of planar surfaces 28 may be positioned in various orientations relative to one another, but one such orientation is in a position where the planar surfaces 28 form an ideal polyhedron when viewed in a cross-section orthogonal to the central axis A, such as is shown in FIG. 2A. The number of planar surfaces 28 may be any number above 2. In various implementations, the number of planar surfaces 28 is determined by the number of corresponding surfaces on the hub of the needle to be bent. In certain implementations, the number of planar surfaces 28 is six. In other implementations, the number of planar surfaces 28 is eight.

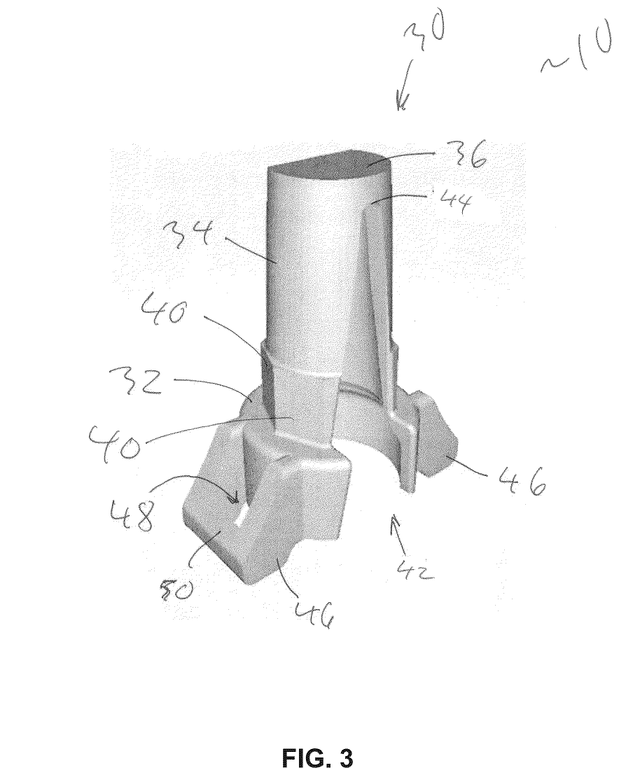

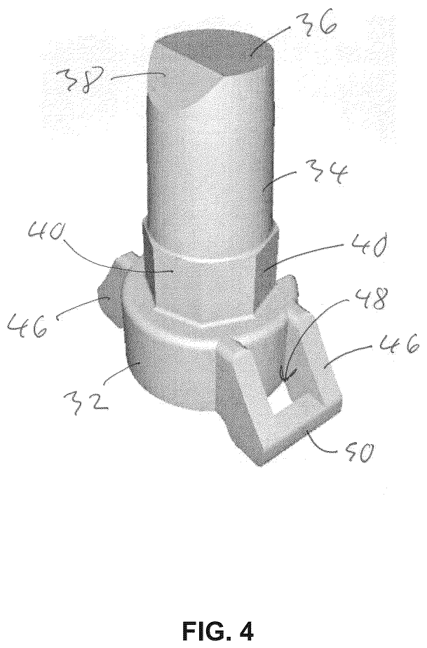

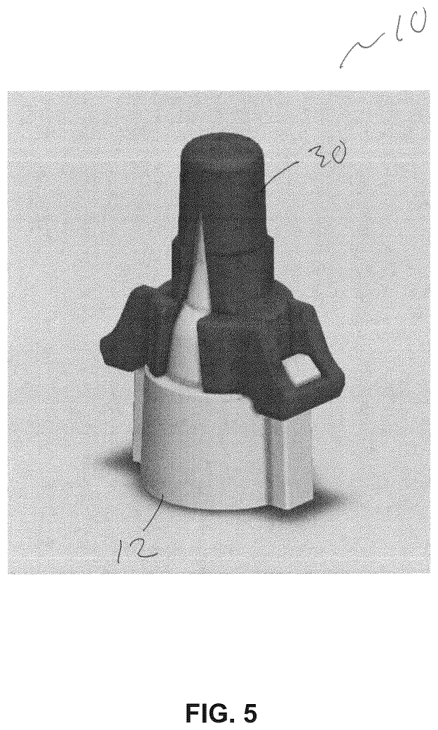

Turning now to FIGS. 3 and 4, the bending device 10 may also have a brace 30. In various implementations, the brace 30 may be made of a lip 32, which may be of a generally cylindrical and hollow shape, and a barrel 34. The barrel 34 may be generally cylindrical or conical and may extend from the lip 32 along the axis of the lip 32. The barrel 34 may be hollow with a top 36 that closes the otherwise cylindrical or conical barrel 34 shape. The top 36, in some implementations, may have an angled portion 38, shown best in FIG. 4, disposed at an angle between the plane of the main portion of the top 36 and the sidewalls of the barrel 34. As would be understood, this angled portion 38 may improve ergonomics and control of the needle in use, as a sharp ridge does not need to be pressed against by the finger of a user.

In various implementations, the barrel 34 may have a plurality of flat planes 40 disposed around the circumference of the barrel 34. The flat planes 40 may be positioned to complement the planar surfaces 28 of the base 12. As would be understood, in implementations where the planar surfaces 28 of the base 12 are positioned to interface with the hub of the needle, the flat planes 40, when made to complement the planar surfaces 28, would be able to couple to the cap of the needle for safe disposal. This ability for mutually coupling will be discussed in more depth below.

In some implementations, such as the one shown in FIG. 3, the brace 30 may have a notch 42 cut into the lip 32 and barrel 34. The notch 42 may be wider when cut into the lip 32 and taper inward through the barrel 34 to a point 44.

In various implementations, the brace 30 may have one or more latches 46. The latches 46 may be protrusions from the lip 32 with an opening 48 through the body of each latch 46. Each latch 46 may have a latch-arm 50 across the opening 48 from the lip 32. In some implementations, the number of latches 46 matches the number of wings 20. In some implementations, the wings 20 may slot into the openings 48 of each latch 46 so that each tab 22 on each wing 20 passes through the latch 46, but cannot be retracted, as the flat surface 24 of each tab 22 rests against the latch-arm 50 of each latch 46. As would be understood, the ability for the tabs 22 to pass through the opening 48 in one direction, but not be able to be retracted depends on the ability of the brace 30 to flex slightly, optionally in the lip 32 and barrel 34. FIG. 5 shows the base 12 and brace 30 fitted together in this manner.

As would be understood, the final angle of the needle may be determined by adjusting the relative position of the point 44 and the top 36 when the base 12 and brace 30 are interlocked. As the base 12 and brace 30 may interlock securely, such that their relative position cannot change, changing the height of the top 36 relative to the wings 20 or changing the depth of the point 44 relative to the latches may adjust the predetermined angle at which the bending device 10 will secure the bent needle. Securing the needle in the point 44 may also stabilize and support the needle after it is bent. Because the needle may rest in the trough of the point 44, it has a physical barrier preventing movement in three directions.

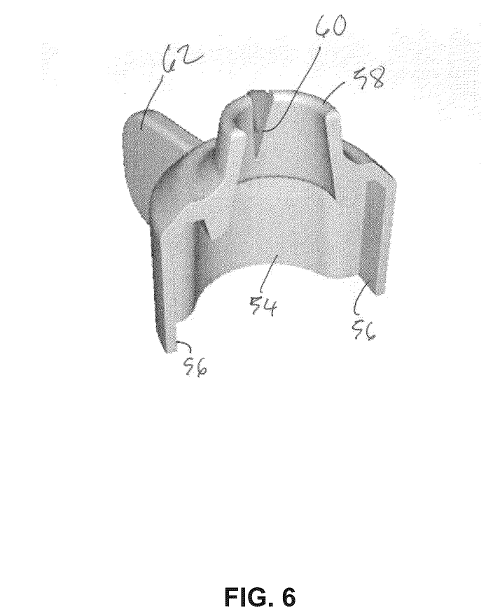

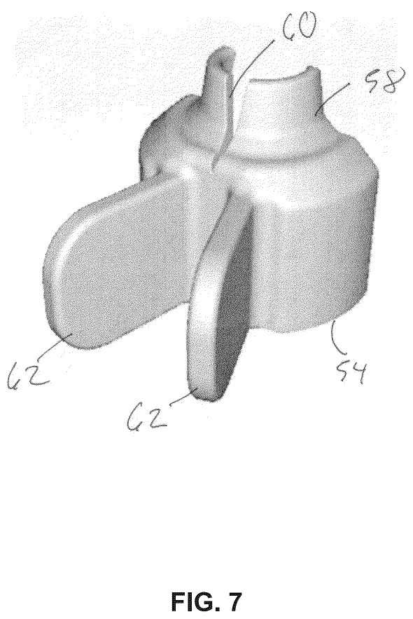

FIGS. 6 and 7 show a handle 52, part of the bending device 10. The handle 52, in various implementations, may have a curved body 54 formed to complement the proximal body 14 and wings 20. The curved body 54 may have two or more flat sides 56 shaped to complement and hold the wings 20. The curved body 54, in some implementations, may extend along the medial body 16 with an upper portion 58. The upper portion 58 may have a slit 60 cut into it. The purpose of the slit will be discussed below.

The handle 52 may also have two or more grips 62 protruding from the curved body 54. The grips 62, in some implementations, may be held by a user and pressed together. When the grips 62 are pressed together, the curved body 54 may be elastically deformed so the flat sides 56 of the curved body 54 are pulled away from one another. In implementations where the slit 60 is disposed between the two grips 62, the reduced amount of material, and the elimination of material not along a single plane, helps to each the effort needed to elastically deform the curved body 54.

The bending device 10 may be employed in a method 100 to ensure successful use. The particular method described herein is merely exemplary and is not the only method possible for use with the bending device 10. All presently disclosed steps are optional and may be rearranged or omitted as desired.

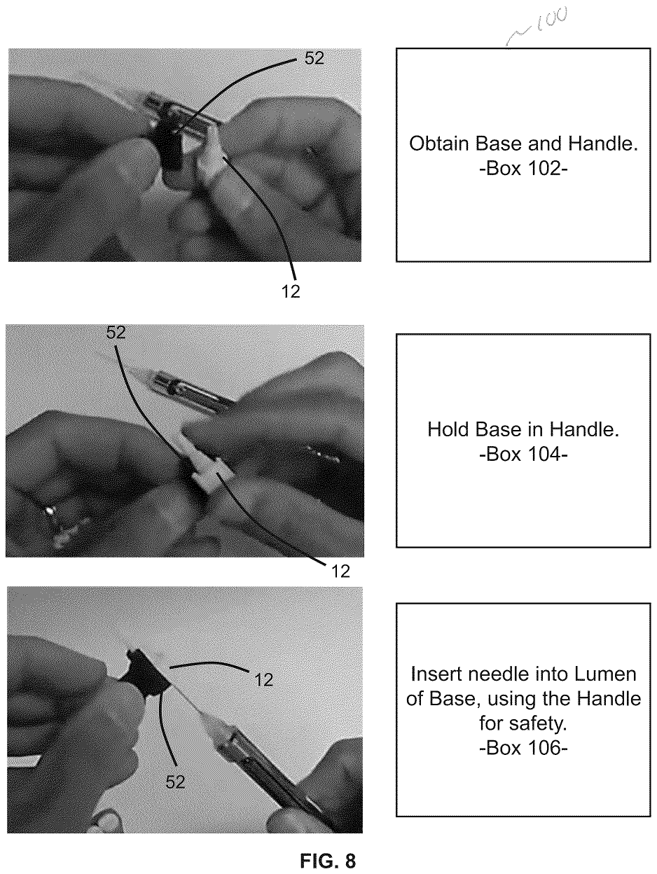

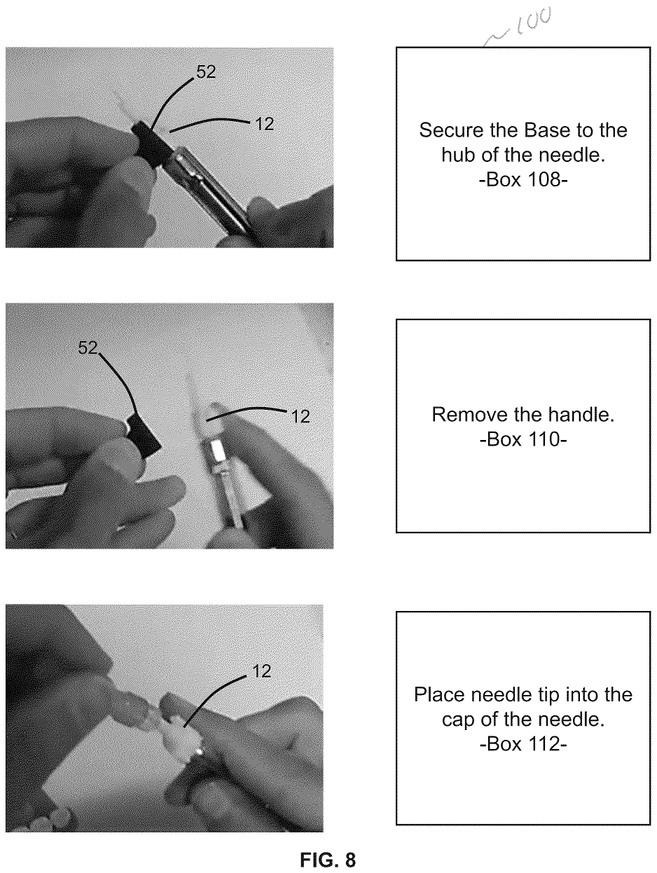

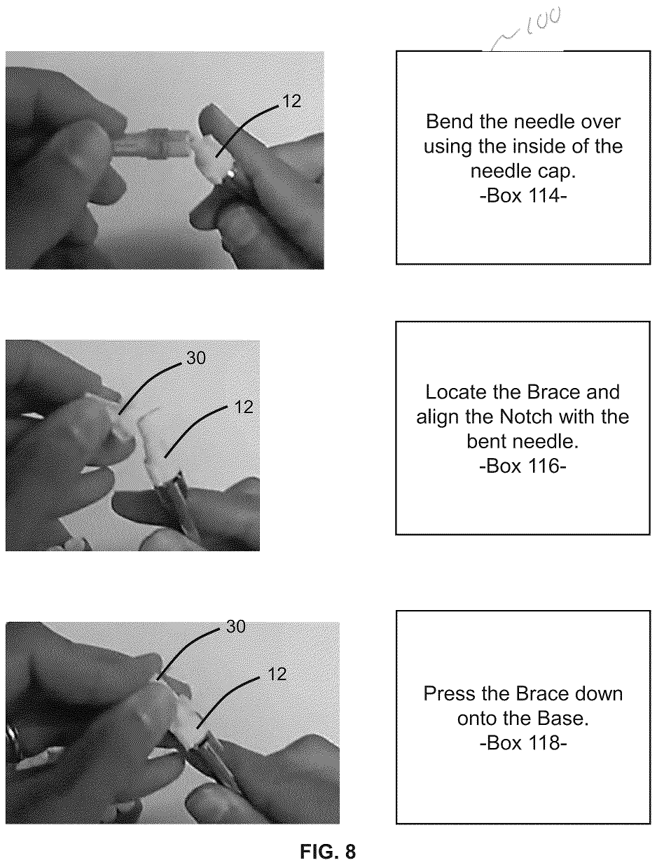

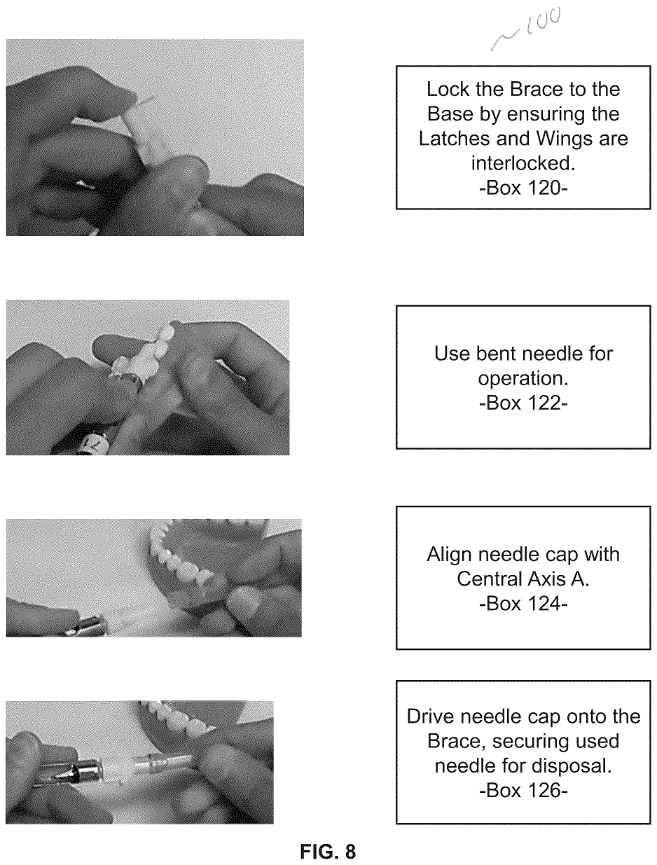

The method 100, according to some implementations such as shown in FIG. 8, may begin by a user obtaining the base 12 and handle 52 (box 102). The user may then hold the base 12 with the handle 52 (box 104). As would be understood, this may be done by compressing the grips 62 to force the flat sides 56 apart, inserting the base 12 into the handle 52, and releasing some pressure on the grips 62 to allow the flat sides 56 to grasp the base 12. The user may then insert the needle into the lumen 26 of the base 12, using the handle 52 to keep their fingers at a safe distance from the needle (box 106). The user may then secure the base 12 onto the hub of the needle by interlocking the planar surfaces 28 with the hub (box 108). The handle 52 may then be removed by compressing the grips 62 to force the flat sides 56 apart, thereby releasing the base 12 from the handle 52 (box 110).

The user may then place the tip of the needle into the cap of the needle (box 112), or in or against any other sterile rigid object. The user may then bend the needle over with the needle (box 114). The exact desired angle need not be achieved at this point.

As would be understood, the cap of the needle would be a convenient sterile object for steps 112 and 114, as essentially all surgical needles are capped until use. However, it is not required that a cap be used, as various objects could be suitable.

The user may locate the brace 30 and align the notch 42 with the bent needle (box 116). The user may press the brace 30 down onto the base 12, whereby the needle travels along the notch 42 toward the point 44 (box 118). The brace 30 may be locked into the base 12 by pressing the brace 30 down far enough for the wings 20 and tabs 22 to pass through the openings 48 so the flat surfaces 24 are locked against the latch-arms 50 (box 120). In this position, the needle is secured at its predetermined angle.

The needle may then be used for its relevant surgical purpose (box 122). Once desired, the cap of the needle may be aligned with the bending device 10, such as axially along the central axis A (box 124). The cap of the needle may then be driven onto the brace 30 (box 126). As would be understood, driving the cap onto the brace 30 may cause the needle to bend such that it is flush with the outer wall of the brace 30. The cap may then pass over the fully bent needle, containing it entirely under itself. The cap may be secured onto the brace 30 due to the flat planes 40 on the brace 30.

Although the disclosure has been described with reference to preferred implementations, persons skilled in the art will recognize that changes may be made in form and detail without departing from the spirit and scope of the disclosed apparatus, systems and methods.

What is claimed is:

1. A needle bending device comprising:
(a) a base with a lumen; and
(b) a brace with a notch,
wherein the base and base are configured to interlock such that the needle is stabilized in the notch.

2. The needle bending device of claim 1, wherein the lumen comprises a plurality of planar surfaces configured to complement a hub of the needle.

3. The needle bending device of claim 1, wherein the brace comprises a plurality of flat planes configured to complement a cap of the needle.

4. The needle bending device of claim 1, wherein base comprises two or more wings.

5. The needle bending device of claim 4, wherein each of the two or more wings comprise a tab.

6. The needle bending device of claim 5, wherein the brace comprises two or more latches.

7. The needle bending device of claim 6, wherein each of the two or more latches comprise an opening and a latch-arm configured to interlock with the tab.

8. The needle bending device of claim 1, further comprising a handle configured to engage with the base, the handle comprising:
a curved body with two or more flat sides; and
two or more grips.

9. The needle bending device of claim 8, wherein the handle further comprises a slit.

10. The needle bending device of claim 1, wherein the brace comprises a lip axially aligned and connected to a barrel.

11. The needle bending device of claim 10, wherein the barrel is hollow.

12. The needle bending device of claim 11, wherein the barrel comprises a notch.

13. The needle bending device of claim 11, wherein the notch comprises a point.

14. The needle bending device of claim 10, wherein the top comprises an angled portion.

15. A system for bending a securing a needle comprising:
(a) a base with a lumen; and
(b) a brace with a notch,
wherein the base is configured to be secured to a hub of a needle, with the needle disposed through the lumen of the base, and
wherein the brace is configured to be secured to the base whereby bending the needle to a predetermined angle.

16. The system of claim 15, wherein the base comprises two or more wings.

17. The system of claim 16, wherein the brace comprises two or more latches.

18. The system of claim 17, wherein the two or more wings are configured to interlock with the two or more latches.

19. The system of claim 15, further comprising a handle configured to hold the base.

20. A medical device for bending a medical needle comprising:
(a) a base with a lumen;
(b) a brace with a notch; and
(c) a handle configured to hold the base to provide additional space between the medical needle and a user,
wherein the base and cap are configured to interlock such that the needle is stabilized in the notch.
